(19) United States
(12) Patent Application Publication
(10) Pub. No.: US 2026/0265456 A1
(43) Pub. Date: Sep. 10, 2026

(54) LIGNIN-BASED BIODEGRADABLE POLYMERS AND METHODS OF MAKING THE SAME

(71) Applicant: The Florida State University Research Foundation, Inc., Tallahassee, FL (US)

(72) Inventor: Hoyong Chung, Tallahassee, FL (US)

(21) Appl. No.: 19/663,565

(22) Filed: Apr. 30, 2026

Related U.S. Application Data
(62) Division of application No. 18/472,645, filed on Sep. 22, 2023.
(60) Provisional application No. 63/408,892, filed on Sep. 22, 2022.

(51) Int. Cl. C08G 64/02; C07D 319/06; C08G 64/30

(52) U.S. Cl. CPC C08G 64/0208; C07D 319/06; C08G 64/30

(57) ABSTRACT

Disclosed are methods of forming lignin-based recyclable polymers. The methods comprise using a carbon dioxide-containing gas as a reagent to form a lignin-based polycarbonate. Also disclosed are methods for capturing carbon dioxide gas and recycling lignin-based materials.

### CROSS REFERENCE TO RELATED APPLICATION

This application is a Divisional Application of U.S. patent application Ser. No. 18/472,645 filed 22 Sep. 2023, which claims priority to and the filing benefit of U.S. Provisional Patent Application 63/408,892 filed 22 Sep. 2022, the contents of which are hereby incorporated in their entireties.

### FIELD

The present disclosure relates generally to lignin based biodegradable polymers that can, for example, be used to replace current petroleum-based plastics and methods for making the same. The present disclosure also relates to manufacturing methods and articles comprising the same.

### BACKGROUND

Plastics have become an inseparable part of modern life. Due to their versatility and continuous use over 300 million tons of plastic material are produced yearly, with about 50% of it for a single purpose. However, the durability of plastics creates disposal problems, and large amounts of plastics are disposed in landfills or dumped into the oceans each year. Conventional plastics take a long time to decompose, which is often accompanied by toxic chemicals being into soil and water. Meanwhile, plastic incineration can result in the production of harmful gases.

The presence of plastics in oceans creates additional problems as it can complicate navigation, entangle and kill marine life, harbor communities of pathogenic bacteria, and leach harmful chemicals into the environment. The presence of microplastics, very small plastic particles that are nearly ubiquitous in water supplies worldwide, is especially problematic as it makes plastic compounds more bioavailable to animals and humans.

Growing consumer environmental awareness, along with industry's desire for cost efficiency, demonstrates a need for plastic materials to have a desirable cradle-to-cradle product life cycle. For example, there is a need for plastic materials made from renewable sources (“bioplastics”) can easily degrade without leaching harmful materials. Currently available bioplastics are not without their drawback. For example, producing biodegradable plastics from shelled corn is not economically efficient, as it requires multi-step processes and competes for human food chain sources.

Lignin is non-human food biomass that is readily available as a byproduct of the biofuel and paper industry. The abundance of aromaticity in lignin is unique compared to other bioplastics, which are composed primarily of aliphatic structures. Lignin can be an excellent renewable resource for producing functional polymers instead of petroleum. In addition, lignin is biodegradable, and lignin-based polymers can be designed to be completely biodegradable. However, the preparation of various polymers from lignin can be complex, involve harsh chemistries, and is not economically valuable.

Environmental concerns are further exacerbated by global warming, which is at least partially caused by the greenhouse effect. CO₂ is a major contributor to the greenhouse effect, and as a result, many attempts are made to reduce CO₂ emissions and increase CO₂ utilization. CO₂ is a cheap and readily available carbon source that can be utilized in many chemical processes, such as synthesizing various polymers.

There remains a need for improved bioplastics and methods for producing them. There remains a need for improved recyclable bioplastics. There remains a need for improved systems and processes for valorizing lignin. There remains a need for improved systems and methods for removing CO₂ from the atmosphere and storing CO₂ in an economically valuable way.

### SUMMARY

The present disclosure is generally methods of making lignin based polymers. In some aspects, such polymers are biodegradable.

In some aspects, disclosed is a method comprising: a) reacting a lignin based material, wherein the lignin based material comprises a plurality of OH group with a gas comprising carbon dioxide to form a lignin-based monomer unit comprising a cyclic carbonate group in the presence of a first catalyst (I), and b) polymerizing the monomer to form a polycarbonate.

In yet other aspects, the first catalyst can comprise 8-diazabicyclo(5.4.0) undec-7-ene (DBU).

In still further aspects, in the methods disclosed herein, the first catalyst can react with at least one OH group out of the plurality of OH groups to form an intermediate product. In such exemplary and unlimiting aspects, the intermediate product can be further reacted with an activating reagent comprising at least one reactive leaving group.

In still further methods, the step of polymerizing comprises a ring opening polymerization.

Also disclosed herein is a method of capturing carbon dioxide, wherein the method comprises: converting carbon dioxide into a polycarbonate material by reaction of a lignin based material with carbon dioxide, wherein the polycarbonate material is fully biodegradable.

In still further aspects, disclosed herein are methods of recycling a lignin-based material, wherein the method comprises: providing a biomass comprising a lignin-based material; and reacting the lignin-based material with carbon dioxide to form a polycarbonate material; wherein the polycarbonate material is fully biodegradable.

Also disclosed herein is a polycarbonate formed by any of the disclosed herein methods.

Also disclosed is an article comprising the polycarbonate material formed by the methods disclosed herein.

Additional aspects of the invention will be set forth, in part, in the detailed description, figures, and claims which follow, and in part will be derived from the detailed description or can be learned by practice of the invention. It is to be understood that both the foregoing general description and the following detailed description are exemplary and explanatory only and are not restrictive of the invention as disclosed.

### DESCRIPTION OF DRAWINGS

FIG. 1A depicts representative structural fragments of lignin.

FIG. 1B depicts various structural moieties found within lignin.

### DETAILED DESCRIPTION

The present invention can be understood more readily by referencing the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

In this specification and in the claims that follow, reference will be made to a number of terms, which shall be defined to have the following meanings:

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of.”

As used herein, the singular forms “a,” “an,” and “the” include plural referents unless the context clearly dictates otherwise. Thus, for example, reference to a “polymer” includes aspects having two or more such polymers unless the context clearly indicates otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

As used herein, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, segment, composition, or other condition for which substantially is used to characterize or otherwise quantify an amount.

In other aspects, as used herein, the term “substantially free,” when used in the context of a composition or segment of a composition that is substantially absent, is intended to refer to an amount that is less than about 1% by weight, e.g., less than about 0.5% by weight, less than about 0.1% by weight, less than about 0.05% by weight, or less than about 0.01% by weight of the stated material, based on the total weight of the composition.

References in the specification and concluding claims to parts by weight of a particular element or component in a composition or article, denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the term “composition” is intended to encompass a product comprising the specified ingredients in the specified amounts, as well as any product which results, directly or indirectly, from a combination of the specified ingredients in the specified amounts.

A weight percent of a segment, unless specifically stated to the contrary, is based on the total weight of the formulation or composition in which the segment is included.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate, effective amount will be readily determined by one of ordinary skill in the art using only routine experimentation.

As used herein, the term “biodegradable” refers to a material capable of being decomposed by bacteria or other living microorganisms.

As used herein, “Kraft lignin” refers to the lignin product of a sulfate pulping process, i.e., the treatment of a biomass (e.g., woodchips) with NaOH and Na₂S. It is understood that Kraft lignin can comprise about 2-3 wt % of sulfur based on the total weight of the Kraft lignin.

As used herein, the terms “modified” and “functionalized” can be used interchangeably.

As used herein, the term “substituted” means that a hydrogen atom is removed and replaced by a substituent. It is contemplated to include all permissible substituents of organic compounds. As used herein, the phrase “optionally substituted” means unsubstituted or substituted. It is to be understood that substitution at a given atom is limited by valency. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein, which satisfy the valencies of the heteroatoms. This disclosure is not intended to be limited in any manner by the permissible substituents of organic compounds. Also, the terms “substitution” or “substituted with” include the implicit proviso that such substitution is in accordance with the permitted valence of the substituted atom and the substituent and that the substitution results in a stable compound, e.g., a compound that does not spontaneously undergo transformation such as by rearrangement, cyclization, elimination, etc. In still further aspects, it is understood that when the disclosure describes a group being substituted, it means that the group is substituted with one or more (i.e., 1, 2, 3, 4, or 5) groups as allowed by valence selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “compound,” as used herein, is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein can also include all isotopes of atoms occurring in the intermediates or final compounds. Isotopes include those atoms having the same atomic number but different mass numbers. For example, isotopes of hydrogen include hydrogen, tritium, and deuterium.

Also provided herein are salts of the compounds described herein. It is understood that the disclosed salts can refer to derivatives of the disclosed compounds wherein the parent compound is modified by converting an existing acid or base moiety to its salt form. Examples of the salts include, but are not limited to, mineral or organic acid salts of basic residues such as amines; alkali or organic salts of acidic residues such as carboxylic acids; and the like. The salts of the compounds provided herein include the conventional non-toxic salts of the parent compound formed, for example, from non-toxic inorganic or organic acids. The salts of the compounds provided herein can be synthesized from the parent compound that contains a basic or acidic moiety by conventional chemical methods. Generally, such salts can be prepared by reacting the free acid or base forms of these compounds with a stoichiometric amount of the appropriate base or acid in water or an organic solvent or in a mixture of the two. In various aspects, nonaqueous media like ether, ethyl acetate, alcohols (e.g., methanol, ethanol, isopropanol, or butanol) or acetonitrile (ACN) can be used.

In various aspects, the compounds provided herein, or salts thereof, are substantially isolated. By “substantially isolated,” it meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

As used herein, chemical structures that contain one or more stereocenters depicted with dashed and bold bonds are meant to indicate the absolute stereochemistry of the stereocenter(s) present in the chemical structure. As used herein, bonds symbolized by a simple line do not indicate a stereo-preference. Unless otherwise indicated to the contrary, chemical structures, which include one or more stereocenters, illustrated herein without indicating absolute or relative stereochemistry encompass all possible stereoisomeric forms of the compound (e.g., diastereomers and enantiomers) and mixtures thereof. Structures with a single bold or dashed line and at least one additional simple line encompass a single enantiomeric series of all possible diastereomers.

The expressions “ambient temperature” and “room temperature” as used herein are understood in the art and refer generally to a temperature, e.g., a reaction temperature, which is about the temperature of the room in which the reaction is conducted, for example, a temperature from about 20° C. to about 30° C.

“R¹,” “R²,” “R³,” “R⁴,” etc., are used herein as generic symbols to represent various specific substituents. These symbols can be any substituents, not limited to those disclosed herein, and when they are defined to be certain substituents in one instance, they can, in another instance, be defined as some other substituents.

The term “hydroxyl,” as used herein, is represented by the formula-OH.

As used herein, the term “amine base” refers to a mono-substituted amino group (i.e., primary amine base), di-substituted amino group (i.e., secondary amine base), or a tri-substituted amine group (i.e., tertiary amine base). Exemplary mono-substituted amine bases include methylamine, ethylamine, propylamine, butylamine, and the like. Examples of di-substituted amine bases include dimethylamine, diethylamine, dipropylamine, dibutylamine, pyrrolidine, piperidine, azepane, morpholine, and the like. In various aspects, the tertiary amine has the formula N(R′)₃, wherein each R′ is independently C₁-C₆ alkyl, 3-10 member cycloalkyl, 4-10 membered heterocycloalkyl, 1-10 membered heteroaryl, and 5-10 membered aryl, wherein the 3-10 member cycloalkyl, 4-10 membered heterocycloalkyl, 1-10 membered heteroaryl, and 5-10 membered aryl is optionally substituted by 1, 2, 3, 4, 5, or 6 Ci-6 alkyl groups. Exemplary tertiary amine bases include trimethylamine, triethylamine, tripropylamine, triisopropylamine, tributylamine, tri-tert-butylamine, N,N-dimethylethanamine, N-ethyl-N-methylpropan-2-amine, N-ethyl-N-isopropylpropan-2-amine, morpholine, N-methylmorpholine, and the like. In various aspects, the term “tertiary amine base” refers to a group of formula N(R)₃, wherein each R is independently a linear or branched C₁₋₆ alkyl group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Dashed lines in a chemical structure are used to indicate that a bond may be present or absent or that it may be a delocalized bond between the indicated atoms.

“Leaving group,” as used herein, refers to a molecule or a molecular fragment (e.g., an anion) that is displaced in a chemical reaction as stable species taking with it the bonding electrons. Examples of leaving groups include an arylsulfonyloxy group or an alkylsulfonyloxy group, such as a mesylate or a tosylate group. Common anionic leaving groups also include halides such as Cl—, Br—, and I—.

As used herein, substantially pure means sufficiently homogeneous to appear free of readily detectable impurities as determined by standard methods of analysis, such as thin-layer chromatography (TLC), nuclear magnetic resonance (NMR), gel electrophoresis, high-performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. Unless specified to the contrary, a substantially chemically pure compound can, however, be a mixture of stereoisomers.

Preparation of the compounds described herein can involve a reaction in the presence of an acid or a base. Example acids can be inorganic or organic acids and include, but are not limited to, strong and weak acids. Example acids include, but are not limited to, hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Example weak acids include, but are not limited to, acetic acid, propionic acid, butanoic acid, benzoic acid, tartaric acid, pentanoic acid, hexanoic acid, heptanoic acid, octanoic acid, nonanoic acid, and decanoic acid. Examples include, without limitation, lithium hydroxide, sodium hydroxide, potassium hydroxide, lithium carbonate, sodium carbonate, potassium carbonate, sodium bicarbonate, and amine bases. Example strong bases include, but are not limited to, hydroxide, alkoxides, metal amides, metal hydrides, metal dialkylamides, and arylamines, wherein; alkoxides include lithium, sodium and potassium salts of methyl, ethyl and t-butyl oxides; metal amides include sodium amide, potassium amide, and lithium amide; metal hydrides include sodium hydride, potassium hydride, and lithium hydride; and metal dialkylamides include lithium, sodium, and potassium salts of methyl, ethyl, n-propyl, isopropyl, n-butyl, t-butyl, trimethylsilyl, and cyclohexyl substituted amides (e.g., lithium N-isopropylcyclohexylamide).

The following abbreviations may be used herein: AcOH (acetic acid); aq. (aqueous); atm. (atmosphere(s)); Br₂ (bromine); Bn (benzyl); calc. (calculated); d (doublet); dd (doublet of doublets); DCM (dichloromethane); DMF (N,N-dimethylformamide); Et (ethyl); Et₂O (diethyl ether); EtOAc (ethyl acetate); EtOH (ethanol); EWG (electron withdrawing group); g (gram(s)); h (hour(s)); HCl (hydrochloric acid/hydrogen chloride); HPLC (high performance liquid chromatography); H₂SO₄ (sulfuric acid); Hz (hertz); (iodine); IPA (isopropyl alcohol); J (coupling constant); KOH (potassium hydroxide); K₃PO₄ (potassium phosphate); LCMS (liquid chromatography-mass spectrometry); GC (gas chromatography), LIICA (lithium N-isopropylcyclohexylamide); m (multiplet); M (molar); MS (Mass spectrometry); Me (methyl); MeCN (acetonitrile); MeOH (methanol); mg (milligram(s)); min. (minutes(s)); mL (milliliter(s)); mmol (millimole(s)); N (normal); NaBH₄CN (sodium cyanoborohydride); NHP (N-heterocyclic phosphine); NHP-C1 (N-heterocyclic phosphine chloride); Na₂CO₃ (sodium carbonate); NaHCO₃ (sodium bicarbonate); NaOH (sodium hydroxide); Na₂SO₄ (sodium sulfate); nM (nanomolar); NMR (nuclear magnetic resonance spectroscopy); PCb (trichlorophosphine); PMP (4-methoxyphenyl); RP-HPLC (reverse phase high performance liquid chromatography); t (triplet or tertiary); t-Bu (teri-butyl); TEA (triethylamine); TFA (trifluoroacetic acid); THF (tetrahydrofuran); TLC (thin layer chromatography); μg (microgram(s)); μL (microliter(s)); μM (micromolar); wt % (weight percent).

Disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, suppose a composition is disclosed, and a number of modifications that can be made to a number of components of the composition are discussed. In that case, each and every combination and permutation that are possible are specifically contemplated unless specifically indicated to the contrary. Thus, if a class of components A, B, and C are disclosed and a class of components D, E, and F and an example of a combination composition A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure, including, but not limited to, steps in methods of making and using the disclosed compositions. Thus, if there are a variety of additional steps that can be performed, it is understood that each of these additional steps can be performed with any specific aspect or combination of aspects of the disclosed methods and that each such combination is specifically contemplated and should be considered disclosed.

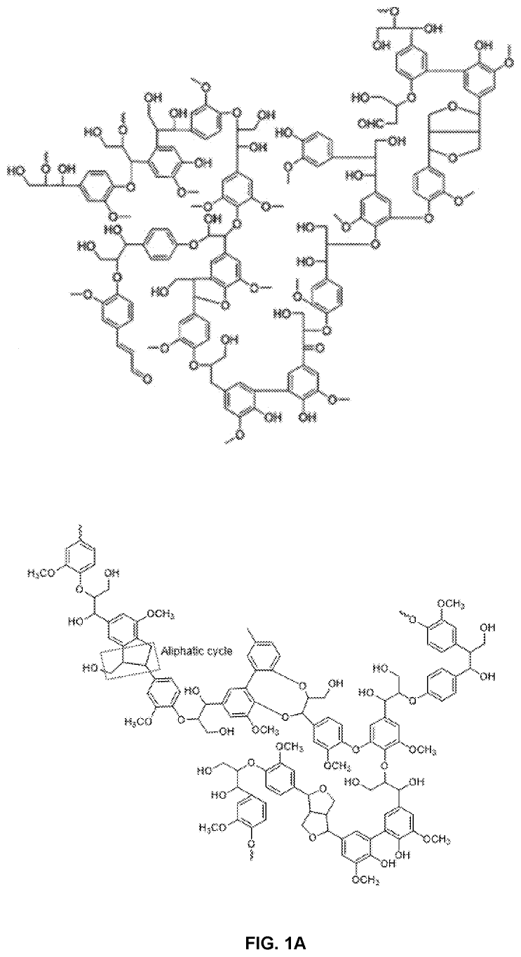

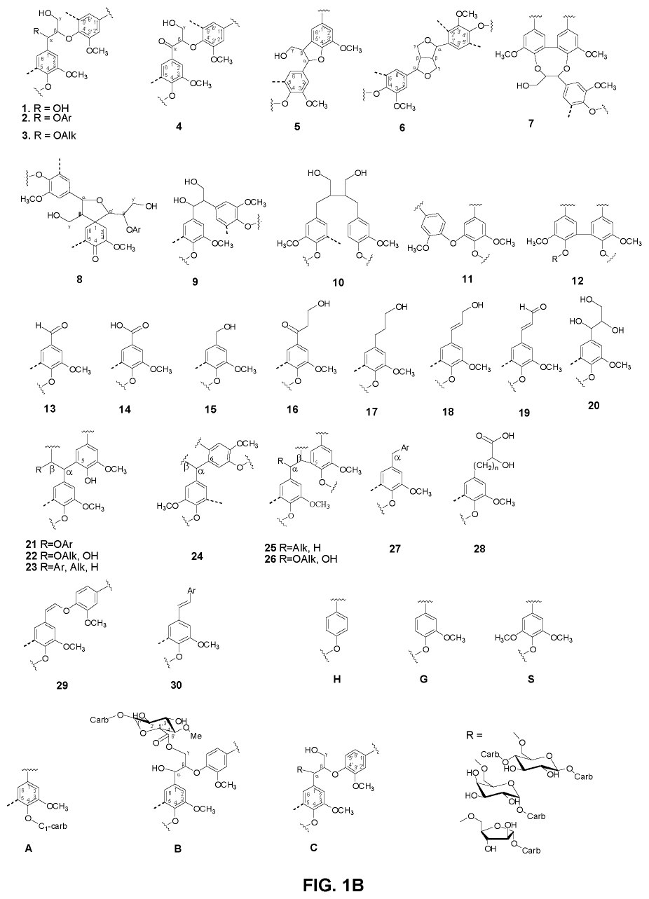

Native lignin is the second most abundant natural polymer on Earth. It is an irregular heterogeneous polymer. FIG. 1A shows an exemplary (partial) lignin structure, depicting various functional groups that can occur within a given lignin sample. The repeatable (monomeric) unit in lignin is the phenylpropane unit (or the so-called C9-unit) of the p-hydroxyphenyl (H), guaiacyl (G) and syringyl(S) types (FIG. 1B). Coniferous lignins are predominantly of the G-type. Hardwood lignins contain both G-units and S-units. The H-unit content in woody lignin is usually low; however, the H-unit content can significantly contribute to the structure of non-woody lignins (for instance, lignins derived from annual fibers). In addition, annual fiber lignins contain significant amounts of cinnamic and ferulic acid derivatives attached to the lignin predominantly by ester linkages with the gamma hydroxyl of the C9-units.

Lignin C9-units can contain different functional groups. The most common functional groups are aromatic methoxyl and phenolic hydroxyl, primary and secondary aliphatic hydroxyls, small amounts of carbonyl groups (of the aldehyde and ketone types) and carboxyl groups. The monomeric C9 lignin units are linked together to form the polymeric structure of lignin via ether and carbon-carbon linkages. The most abundant lignin inter-unit linkage is the β-O-4 type of linkage (see structures 1-4 and 7 of FIG. 1B). They constitute about 50% of the inter-unit linkages in lignin (about 45% in softwoods, and up to 60-65% in hardwoods). Other common lignin inter-unit linkages are the resinol (β-β) (structure 6), phenylcoumaran (3-5) (structure 5), 5-5 (structure 12) and 4-O-5 (structure 11) moieties. Their number varies in different lignins but typically does not exceed 10% of the total lignin moieties. The number of other lignin moieties is usually below 5%.

The degree of lignin condensation (“DC”) is an important lignin characteristic, as it is often negatively correlated with lignin reactivity. Most commonly, condensed lignin structures are lignin moieties linked to other lignin units via the 2, 5 or 6 positions of the aromatic ring (in H-units also via the C-3 position). The most common condensed structures are 5-5′, B-5 and 4-O-5′ structures. Since the C-5 position of the syringyl aromatic ring is occupied by a methoxyl group, and therefore it cannot be involved in condensation, hardwood lignins are typically less condensed than softwood lignins.

Technical lignins are obtained as a result of lignocellulosic biomass processing. Technical lignins are more heterogeneous (in terms of a chemical structure and molecular mass) than native lignins. Technical lignins can have a higher amount of phenolic hydroxyls than native lignin and have a smaller molecular weight. Technical lignins can have a smaller amount of aliphatic hydroxyls, oxygenated aliphatic moieties and the formation of carboxyl groups and saturated aliphatic structures. The actual structure of technical lignins also depends on the specific biomass processing (acidic vs. basic, and the like).

Lignins suitable for the disclosed processes include those having 1,3 diol fragments:

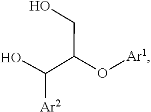

wherein Ar¹ and Ar² are independently an aromatic ring in a lignin structural moiety, for example as depicted in FIG. 1b such as any of moieties 1-30. When Ar¹ and Ar² are lignin structural moieties as shown in FIG. 1b, the dashed line indicates a point of attachment to the fragment above. The skilled person understands that other undefined substituents may be selected from H, CH₃, or another lignin structural moiety. By way of example, one such 1,3 diol fragment that has been observed has the formula:

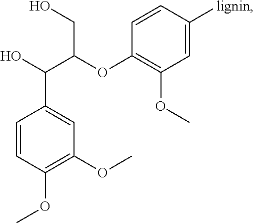

wherein “lignin” represents one or more additional phenyl propane unit as described above. Other 1,3 diol moieties are depicted in FIG. 1B as moieties 1, 9, and 20. In certain implementations, the 1,3 diol moieties does not include fragments that also include a hydroxyl group at the relative 2 position (e.g., moiety 20 in FIG. 1B).

Unless specifically stated to the contrary, the use of an exemplary lignin fragment such as shown above is not intended to limit the disclosed processes, monomers, and polymers to the specifically depicted substitution pattern.

The large abundance of lignin makes it a unique material to be used as a source of other biodegradable polymers. It is understood that the present disclosure is not limited to any specific types of lignin. In certain aspects, the lignin used in the current disclosure can be obtained from natural lignin products or synthetic model lignin compounds. In still further aspects, lignin used in the current disclosure can be obtained from natural lignin products. It is understood that the natural lignin product can comprise softwood lignin, hardwood lignin, or a combination thereof. In certain aspects and without limitations, the natural lignin product can be obtained from agricultural residues (including corn stover and sugarcane bagasse), (2) dedicated energy crops, (3) wood residues (including sawmill and paper mill discards), and (4) municipal waste, and their constituent parts. In still further aspects, the natural lignin product can be obtained from the paper industry. In certain aspects, lignin used herein can comprise Kraft lignin and lignosulfonate. In certain implementations, the lignin can be a dealkaline lignin. In certain implementations, the lignin can have a weight average molecular weight (M_{w}) from 10,000-25,000 g/mol, 25,000-50,000 g/mol, 10,000-50,000 g/mol, 1,000-10,000 g/mol, from 1,000-5,000 g/mol, from 1,000-2,000 g/mol, from 1,000-3,000 g/mol, from 1,000-4,000 g/mol, from 2,000-5,000 g/mol, from 2,000-4,000 g/mol, from 2,000-3,000 g/mol, from 3,000-5,000 g/mol, or from 4,000-5,000 g/mol. In certain implementations, the lignin can have a number average molecular weight (Mₙ) from 500-2,000 g/mol, from 500-1,000 g/mol, from 500-750 g/mol, from 750-1,000 g/mol, from 1,000-1,250 g/mol, from 1,000-1,500 g/mol, from 1,250-1,750 g/mol, from 1,250-1,500 g/mol, from 1,500-2,000 g/mol, from 1,500-1,750 g/mol, or from 1,750-2,000 g/mol. In certain implementations, the lignin can have polydispersity index (PDI M_{w}/Mₙ) from 1-5, from 2-5, from 3-5, from 4-5, from 1-1.5, from 1.5-2 from 1-2, from 1-3, from 1-4, from 2-5, from 2-4, from 2-3, from 2-2.5, from 2.5-3, from 3-5, from 3-4, from 3-3.5, from 3.5-4, from 4-4.5, from 4.5-5, or from 4-5. In certain implementations, the molecular weights can be determined using HPLC, in some implementations the molecular weights can be determined using GPC.

The current disclosure is directed to the methods of making polymeric materials from biomass lignin and CO₂ gas. As discussed, both lignin and carbon dioxide are abundant and low-cost materials that can be utilized to form biodegradable polymers in a more environmentally friendly way.

In one aspect, disclosed is a method comprising: a) reacting a lignin-based material, wherein the lignin-based material comprises a plurality of OH groups with carbon dioxide to form a lignin-based monomer unit comprising a six-atom carbonate group in the presence of a first catalyst. The method can further include the step of polymerizing the monomer to form a polycarbonate. In some aspects, the lignin based material includes one or more of the 1,3 diol functional groups depicted above.

In certain exemplary and non-limiting aspects, the first catalyst can comprise a cerium catalyst like CeO₂ or CeO₂—ZrO (mixite) oxide catalysts, a tin catalyst such as Bu₂Sn(OCH₃)₂, an organometallic catalyst such as copper-based catalysts, magnesium-based catalysts, aluminum-based catalysts, silver-based catalysts, for example, AgOAc, zinc-based catalysts, such as ZnI₂/NEt₃, cobalt-based catalysts, chromium-based catalysts, iron-based catalysts, nickel-based catalysts, titanium-based catalysts, hafnium-based catalysts, and zirconium-based catalysts, a tertiary amine catalyst such as 1,8-diazabicyclo(5.4.0) undec-7-ene (DBU), or any combination thereof. In yet still further aspects, the first catalyst comprises 1,8-diazabicyclo(5.4.0) undec-7-ene (DBU):

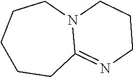 [SMILES: C1CCC2=NCCCN2CC1]

In still further aspects, the reaction can be performed at a pressure of about 1 atm to about 5 atm, about 1 atm to 4 atm, 1 atm to 3 atm, 1 atm to 2 atm, 2 atm to 5 atm, 2 atm 4 atm, 2 atm to 3 atm, 3 atm to 5 atm, or 4 atm to 5 atm, including exemplary values of about 1.5 atm, about 2 atm, about 2.5 atm, about 3 atm, about 3.5 atm, about 4 atm, and about 4.5 atm. The skilled person understands that reactions performed at pressure greater than about 1 atm are conducted in an appropriately sealed vessel. The reaction can be performed using substantially pure carbon dioxide gas or with air, including compressed air.

In still further aspects, the step of reacting is performed at a temperature from about 20° C. to about 35° C., including exemplary values of about 21° C., about 22° C., about 23° C., about 24° C., about 25° C., about 26° C., about 27° C., about 28° C., about 29° C., about 30° C., about 31° C., about 32° C., about 33° C., and about 34° C. In yet still further aspects, the step of reacting is performed at room temperature. In certain implementations, the reaction is conducted at a temperature from 10-100° C., from 10-50° C., from 10-25° C., from 25-50° C., from 20-35° C., from 30-50° C., from 40-60° C., or from 50-100° C.

In yet still further aspects, the step of reacting is performed for about 1 h to about 5 h, including exemplary values of about 1.2 h, about 1.5 h, about 1.8 h, about 2 h, about 2.2 h, about 2.5 h, about 2.8 h, about 3 h, 3.2 h, about 3.5 h, about 3.8 h, about 4 h, about 4.2 h, about 4.5 h, and about 4.8 h. In certain implementations, the reaction is conducted for a period of time from 2-5 h, from 3-5 h, from 4-5 h, from 1-4 h, from 2-4 h, from 3-4 h, from 3-5 h, from 3-4 h, or from 4-5 h.

In some implementations, the catalyst can be present in an amount (relative to the amount of the lignin) that is from 0.01-10 wt. %, from 0.01-1 wt. %, from 0.01-0.1 wt. %, from 0.1-0.25 wt. %, from 0.1-0.5 wt. %, form 0.25-0.5 wt. %, from 0.1-1 wt. %, from 0.5-1 wt. %, from 1-2.5 wt. %, from 1-5 wt. %, or from 2.5-5 wt. %.

In some aspects, the first catalyst reacts with at least one OH group out of the plurality of OH groups to form an intermediate product (I). An exemplary reaction scheme is shown below (Scheme 1):

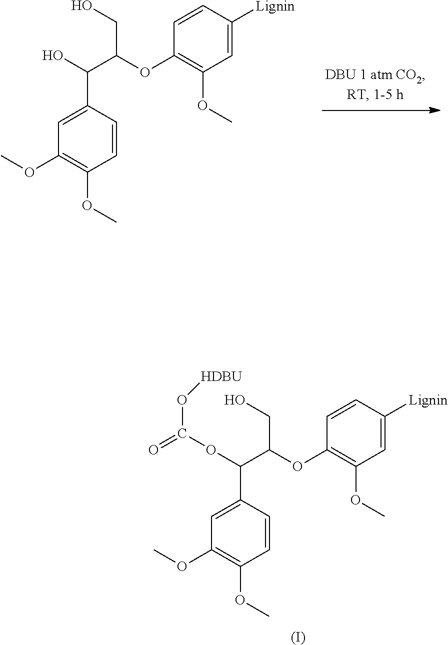

The skilled person understands that additional reactive intermediates may be formed, for instance a carbonic acid may be installed on the primary alcohol group. The various intermediates are in chemical equilibrium, which may be driven towards the carbonic acid with an excess of carbon dioxide. Unless specified to the contrary, the depiction of one exemplary chemical intermediate does not exclude the presence of other intermediates.

In still further aspects, the intermediate product (I) can be further reacted with an activating reagent comprising at least one reactive leaving group to give an activated intermediate. In some aspects, the activating reagent can include a dihalide, aryl or alkyl halide, sulfonyl halide, acid chloride, or any combination thereof, optionally in combination with a base. In certain implementations the activating reagent is a sulfonyl halide like methane sulfonyl chloride, trifluoromethanesulfonyl chloride, or toluenesulfonyl (“tosyl”) chloride. (“TsCl”) and a tertiary amine, for example triethylamine or diethylisopropylamine.

In still further aspects, the intermediate product is reacted with the activating reagent at a temperature from 0° C. to about 35° C., including exemplary values of about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about ° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., and about 34° C. In some implementations, the intermediate product is reacted with the activating reagent at a temperature from 0-25° C., from 0-20° C., from 0-15° C., from 0-10° C., from 10-30° C., from 10-25° C., from 10-20° C., from 15-25° C., from 20-50° C., from 20-30° C., from 30-50° C., or from 25-35° C.

In still further aspects, the intermediate product is reacted with the activating reagent for about 12 h to about 24 h, including exemplary values of about 13 h, about 14 h, about 15 h, about 16 h, about 17 h, about 18 h, about 19 h, about 20 h, about 21 h, about 22 h, and about 23 h. In some implementations, the intermediate product is reacted with the activating reagent for a time that is from 6-48 h, from 12-48 h, from 18-48 h, from 24-48 h, from 6-12 h, from 6-18 h, from 12-24 h, or from 18-36 h.

The exemplary reaction between the intermediate product (I) and the activating reagent is shown in Scheme (2). In this example, the intermediate product (I) is reacted with TsCl/NEts to form an additional intermediate product (II) that then forms a lignin-based monomer unit (III) comprising a carbonate group. In certain aspects, the carbonate group is a cyclic carbonate group, for example a six-atom cyclic carbonate or a five-atom cyclic carbonate. In preferred implementations, the carbonate is a six-atom cyclic carbonate.

In certain implementations the reaction is carried out in a solvent selected from dimethylacetamide, dimethylformamide, acetonitrile, dimethylsulfoxide, or a combination thereof. In certain implementations, the dielectric constant (ε) (at 20° C.) of the solvent is at least 25, at least 30, or at least 35. It has been found that the use of a solvent with similar dielectric constant to the cyclic carbonate (64) drives the equilibrium towards the cyclic carbonate monomer. In certain implementations, the reaction is carried out in a non-protic solvent (for example, a solvent other than water, alkyl alcohols, or carboxylic acids) as these solvents can compete with formation of the cyclic monomer.

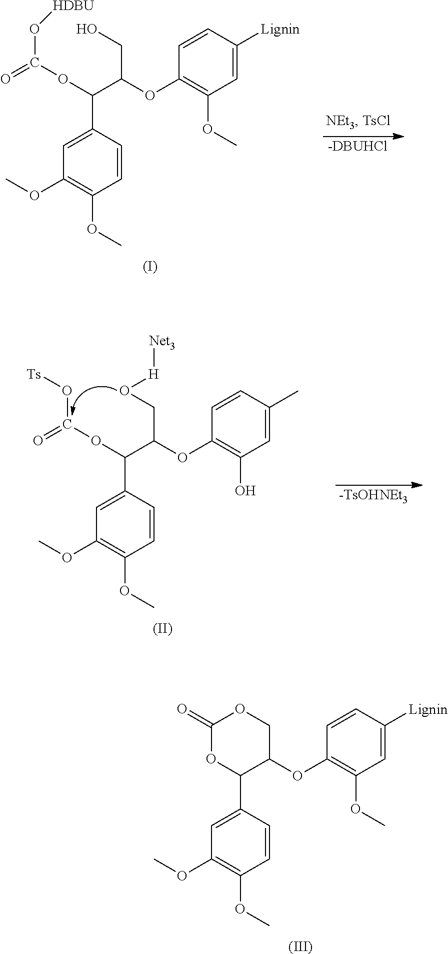

As disclosed herein are lignin derived cyclic six-atom carbonates having the formula:

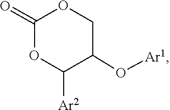

wherein Ar¹ and Ar² are as defined above. The cyclic carbonate may be obtained according to the processes disclosed herein.

In some aspects, the lignin-based monomer unit (III) comprising a six-atom carbonate group is polymerized to form a polycarbonate. In such exemplary and unlimiting aspects, the step of polymerizing comprises a ring opening polymerization (ROP).

In certain aspects, the ring opening polymerization can be performed in the presence of the second catalyst, and initiator, or a combination thereof. As used herein, the second catalyst is a compound that is not ultimately incorporated into the polycarbonate system. An initiator, on the other hand, is typically incorporated at the carboxyl terminus of the polycarbonate.

In certain implementations, the second catalyst includes an organometallic compound, for example catalysts comprising a metal including Al, Sb, Sn, Mg, Ca, Fe, Zn, Zr, or Ln, a Lewis acid, e.g., a compound having the formula MXₙ wherein M is a metal, X is a ligand, and n is a number from 1-6 selected to satisfy valence. In some cases, M is Al, Mg, Ca, B, Fe, Sc, Y, Sr, Ba, Co, Cr, Ir, Ga, Ti, V, Ce, Sm, Zr, Zn, Sn, or Yb, X can be chosen from OTs, OTf, OMs, OAC, Cl, Br, F, or I, and n is equal to the oxidation state of M. It is understood that any other known in the art Lewis acids can also be used a Bronsted acid (for example a sulfonic acid, phosphonic acid, or carboxylic acid), or an amine, for example a guanidine, amidine, or tertiary amine. The second catalyst can include Sb₂O₃, Sb₂O₅, antimony acetate, dodecylbenzenesulfonic acid, methanesulfonic acid, trifluoromethanesulfonic acid, trifluoroacetic acid, toluenesulfonic acid, diphenyl phosphate, 1,5,7-triazabicyclo[4.4.0]dec-5-ene (TBD), 1,2,3-tricyclohexylguanidine, 1,2,3-triisopropylguanidine, or any combination thereof. In still further aspects, the catalyst can be present in any amount. In still further aspects, the second catalyst comprises 1,5,7-triazabicyclo[4.4.0]dec-5-ene (TBD).

In still further aspects, the ring opening polymerization is performed in the presence of an initiator. It is understood that any known in the art of ROP initiators that are suitable for the desired application can be utilized. In certain aspects, the initiators can comprise one or more alcohols or carboxylic acids, (for instance a phenol, benzyl alcohol), or any combination thereof.

In some aspects and as disclosed herein, the catalyst and the initiator can be the same or different.

In still further aspects, any of the disclosed herein, the ROP reaction can be performed at a temperature range from about 10° C. to about 180° C., including exemplary values of about 20° C., about 30° C., about 40° C., about 50° C., about 60° C., about 70° C., about 80° C., about 90° C., about 100° C., about 110° C., about 120° C., about 130° C., about 140° C., about 150° C., about 160° C., and about 170° C. In certain implementations, the ROP reaction is performed at room temperature. In certain implementations the ROP reaction can be performed at a temperature of at least about 20° C., at least about 30° C., at least about 40° C., at least about 50° C., at least about 60° C., at least about 70° C., at least about 80° C., at least about 90° C., at least about 100° C., at least about 110° C., at least about 120° C., at least about 130° C., at least about 140° C., at least about 150° C., at least about 160° C., or at least about 170° C. In certain implementations, the ROP reaction is performed at a temperature from 10-35° C., from 15-30° C., from 20-30° C., or from 20-25° C.

In yet further aspects, the ROP reaction is performed for a time that is from about 5 sec to 5 hours, including exemplary values of about 10 sec, about 30 sec, about 1 min, about 10 min, about 30 min, about 1 hour, about 2 hours, about 3 hours, and about 4 hours. In certain implementations, the ROP reaction is performed for a time between 5-600 minutes, from 5-400 minutes, from 5-250 minutes, from 5-100 minutes, from 5-50 minutes, or from 25-50 minutes. In still other aspects, the time period needed to complete the disclosed herein ROP reactions can be any time needed to provide a desirable amount of the product.

In still further aspects, the ROP can be performed in the presence of a solvent. Any known in the art solvents can be used, for example, organic solvents, inorganic solvents, ionic liquids, and the like. In certain implementations, the ROP reaction can be performed in a solvent having a dielectric constant (ε) (at 20° C.) that is no greater than 25, no greater than 20, no greater than 15, no greater than 10, or no greater than 5. In certain implementations, the ROP reaction can be performed in a solvent having a dielectric constant (ε) (at 20° C.) that is from 0-25, from 0-20, from 0-15, from 1-20, from 1-15, from 1-10, from 1-7.5, from 1-5, or from 5-10. In certain implementations, the second solvent is dichloromethane, toluene, ethyl acetate, n-butyl acetate, glyme, methylethylketone, acetone, or a combination thereof.

Also disclosed herein are lignin derived polycarbonates having the formula:

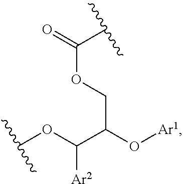

wherein Ar¹ and Ar² are as defined above. The lignin derived polycarbonates can be obtained according to the processes disclosed herein.

In still further exemplary aspects, the formation of polycarbonate through ring opening polymerization can occur according to Scheme (3).

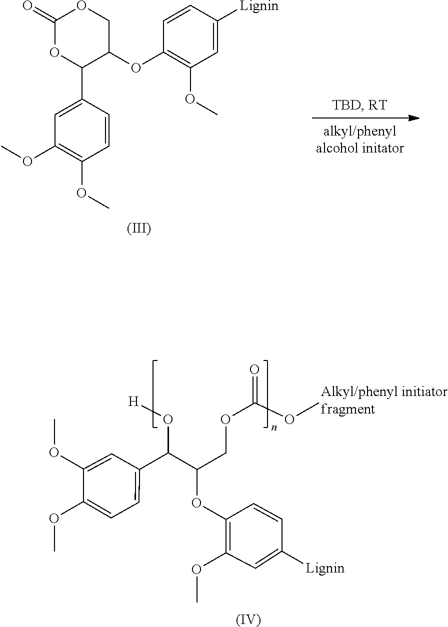

It is understood that the polycarbonate can have any desired molecular weight. For example, the molecular weight of the polycarbonate can range from about 1,000 Da to about 200,000 Da, including exemplary values of about 5,000 Da, about 10,000 Da, about 15,000 Da, about 20,000 Da, about 25,000 Da, about 30,000 Da, about 35,000 Da, about 40,000 Da, about 45,000 Da, about 50,000 Da, about 55,000 Da, about 60,000 Da, about 65,000 Da, about 70,000 Da, about 75,000 Da, about 80,000 Da, about 85,000 Da, about 90,000 Da, about 95,000 Da, about 100,000 Da, about 105,000 Da, about 110,000 Da, about 115,000 Da, about 120,000 Da, about 125,000 Da, about 130,000 Da, about 135,000 Da, about 140,000 Da, about 145,000 Da, about 150,000 Da, about 155,000 Da, about 160,000 Da, about 165,000 Da, about 170,000 Da, about 175,000 Da, about 180,000 Da, about 185,000 Da, about 190,000 Da, about 195,000 Da. In some implementations, the polycarbonate can have a number average molecular weight (as measured by GPC) that is from 50-500 kDa, from 100-500 kDa, from 100-250 kDa, from 100-200 kDa, from 150-200 kDa, from, from 100-150 kDa, from 125-150 kDa, from 100-125 kDa, from 150-175 kDa, from 175-200 kDa. In some implementations, the polycarbonate can have a weight average molecular weight (as measured by GPC) that is from 50-500 kDa, from 100-500 kDa, from 100-300 kDa, from 200-300 kDa, from 150-200 kDa, from 150-175 kDa, from 175-200 kDa, from 150-250 kDa, from 200-250 kDa, from 200-225 kDa, from 200-250 kDa, or from 225-250 kDa. In certain implementations, the polycarbonate can have a polydispersity from 1-2, from 1-1.5, 1-1.25, from 1.25-1.5, from 1.5-1.75, or from 1.75-2.

In still further aspects, the formed polycarbonate is substantially biodegradable. Yet, in other aspects, the formed polycarbonate is fully biodegradable.

The polycarbonate may be recycled to the cyclic carbonate monomer by treatment a recycling catalyst in a recycling solvent. In certain exemplary and non-limiting aspects, the recycling catalyst includes a cerium catalyst like CeO₂ or CeO₂—ZrO (mixite) oxide catalysts, a tin catalyst such as Bu₂Sn(OCH₃)₂, an organometallic catalyst such as copper-based catalysts, magnesium-based catalysts, aluminum-based catalysts, silver-based catalysts, for example, AgOAc, zinc-based catalysts, such as ZnI₂/NEt₃, cobalt-based catalysts, chromium-based catalysts, iron-based catalysts, nickel-based catalysts, titanium-based catalysts, hafnium-based catalysts, and zirconium-based catalysts, a tertiary amine catalyst such as 1,8-diazabicyclo(5.4.0) undec-7-ene (DBU), or any combination thereof.

In certain implementations the recycling is carried out in a solvent selected from dimethylacetamide, dimethylformamide, acetonitrile, dimethylsulfoxide, or a combination thereof. In certain implementations, the dielectric constant (ε) (at 20° C.) of the recycling solvent is at least 25, at least 30, or at least 35.

Also disclosed herein is a method of capturing carbon dioxide, wherein the method comprises: converting carbon dioxide into a polycarbonate material by reaction of a lignin-based material with carbon dioxide; wherein the polycarbonate material is fully biodegradable. In such aspects, the method ensures the cradle-to-cradle formation of fully biodegradable polycarbonate and reduces carbon dioxide emissions.

Also disclosed is a method of recycling a lignin-based material, which comprises: providing a biomass comprising a lignin-based material; and reacting the lignin-based material with a stream of carbon dioxide to form a polycarbonate material; wherein the polycarbonate material is fully biodegradable.

It is understood that the polycarbonate is completely degradable by enzyme/bacteria and hydrolysis, which are commonly available in the natural environment.

The polycarbonate exhibits high mechanical strength and thermal properties. In certain implementations, the polycarbonate has a glass transition temperature (T_{g}) from 30-60° C., from 30-45° C., from 45-60° C., from 30-40° C., from 35-45° C., from 40-50° C., from 45-55° C., or from 50-60° C.

Also disclosed are articles comprising the polycarbonate material formed by the disclosed herein methods. For example, and without limitations, the articles comprising the disclosed herein polycarbonates can comprise components of optical storage devices, automotive parts, packaging, electronics, mirrors and the like. In still further aspects, the articles formed herein can be used in the field of medicine, bioengineering electronics, textile, containers, furniture, automotive, military equipment, coatings, appliances, films, and the like. In certain aspects, the articles prepared from the disclosed biodegradable lignin-based polymers can also comprise packaging, food packaging, disposable cutlery, tableware, film, bags, nets, or any combination thereof.

In still further aspects, disclosed are methods of making the articles, wherein the methods can comprise a step of extrusion, compression molding, injection molding, transfer molding, blow molding, or any combination thereof.

### EXAMPLES

The following examples are for the purpose of illustration of the invention only and are not intended to limit the scope of the present invention in any manner whatsoever.

### Example 1—Synthesis of Cyclic Carbonate Monomer from Lignin and CO₂

Three grams of lignin was placed into a dry three-neck round bottom flask furnished with two dropping funnels, and a magnetic stirrer. Anhydrous dimethylformamide (DMF) (0.1 mol L⁻¹, 135 mL) was added to the sealed flask and stirred until it formed a homogeneous dark brown solution. The system was purged of air by continuous argon gas flow to create a closed inert atmosphere. Then, a carbon dioxide (CO₂) gas-filled balloon was inserted into the system to replace the argon gas with CO₂. The reaction mixture was saturated with CO₂ for 10 minutes and 2.02 mL of DBU (13.5 mmol) was added dropwise into the CO₂-infused saturated solution of lignin in DMF. The reaction mixture became viscous after the addition. After being stirred for 2 hours at room temperature, the flask was placed in an ice bath to cool down the temperature to 0° C. Subsequently, CO₂-saturated 1.88 mL triethylamine (TEA) (13.5 mmol) was added gradually to the solution followed by the slow addition of CO₂-saturated TsCl (2.57 g, 13.5 mmol) solution in DMF. Both the TEA and TsCl solutions were added in a dropwise manner using the attached dropping funnels. Afterward, the reaction mixture was allowed to come to room temperature and stirred for an additional 24 hours. The crude product was washed several times with deionized water, DCM, and brine solution. The organic layer was collected and passed through the anhydrous MgSO₄ to eliminate the trapped water. Lastly, a dark brown sticky product was obtained after evaporating the organic layer using a rotary evaporator. The total yield was 1.92 g (64 wt %).

### Example 2—Ring Opening Polymerization

The following procedure for the preparation of polymer P-4 is representative of the ring opening polymerization. The cyclic carbonate monomer (410 mg, OH content: 4.5 mmol g⁻¹, 1 equivalent) was taken in a pressure relief cap-containing 20 mL vial with a magnetic stirrer. 1.8 mL (1 mol L⁻¹) of anhydrous DCM was added in the vial and continuously flushed with argon gas. A solution of TBD (2.57 mg, 0.018 mmol, 0.01 equivalent) was prepared in anhydrous dichloromethane (DCM) with a concentration of 1 mol L⁻¹. Then, 18 μL of TBD solution was added in the monomer solution under argon atmosphere. The reaction mixture was stirred at room temperature for 30 minutes. The samples were collected in aliquots to track the polymer formation by ¹H NMR spectroscopy and GPC. Subsequently, the collected reaction solutions were quenched with benzoic acid. After removing the solvent under reduced pressure, the crude product was dissolved in the least quantity of DCM and precipitated from diethyl ether. Multiple washes with diethyl ether yielded a light yellowish product.

The reaction time (2, 5, 10, and 30 min) and amount of TBD (0.5, 1.0, 2.0, and 5.0%) were varied for different polymerization reactions:

TABLE 1

| | | | Time | Mₙ^{b} | M_{w}^{c} | | T_{d5%}^{e} | T_{g}^{f} |
|---|---|---|---|---|---|---|---|---|
| Polymer | Cat. | [M]:[C]^{a} | (Min) | (kDa) | (kDa) | Ð^{d} | (° C.) | (° C.) |
| P-1 | TBD | 100:1 | 2 | 123.22 | 190.47 | 1.55 | 244 | 45 |
| P-2 | TBD | 100:1 | 5 | 125.78 | 201.17 | 1.60 | 257 | 47 |
| P-3 | TBD | 100:1 | 10 | 144.78 | 189.46 | 1.31 | 259 | 50 |
| P-4 | TBD | 100:1 | 30 | 154.58 | 229.34 | 1.48 | 277 | 52 |
| P-5 | TBD | 100:2 | 30 | 141.04 | 211.20 | 1.50 | 253 | 46 |
| P-6 | TBD | 100:5 | 30 | 129.88 | 205.06 | 1.58 | 255 | 43 |
| P-7 | TBD | 200:1 | 30 | 139.50 | 223.01 | 1.60 | 260 | 48 |
| P-8 | DBU | 100:1 | 40 | 128.87 | 191.44 | 1.49 | 265 | 47 |
| P-9 | DBU | 100:2 | 40 | 122.93 | 182.38 | 1.48 | 263 | 40 |
| P-10 | DBU | 100:5 | 40 | 120.34 | 229.22 | 1.90 | 253 | 33 |

^{a}monomer: catalyst ratio;
^{b}number-average molecular weight;
^{c}weight-average molecular weight;
^{d}polydispersity index;
^{e}5% decomposition temperature in TGA;
^{f}glass transition temperature from DSC.

### Example 3—Chemical Recycling

The chemical recycling process of P-4 to the cyclic monomer was elaborated as follows. P-4 (250 mg, OH content: 4.5 mmol g⁻¹, 1 equivalent), DBU (8.56 mg, 0.05 equivalent) and 2.2 mL of acetonitrile (0.5 mol L⁻¹) were taken in a 4 mL vial charged with a magnetic stirrer. After attaining a homogeneous brown solution, the reaction mixture was placed in an oil bath at 90° C. and stirred for 24 hours. Afterward, the temperature cooled down to room temperature. Rotary evaporation was used to concentrate the crude solution. Subsequently, the product was washed several times with DCM and water. Finally, a dark brown sticky substance was obtained after concentrating the filtered organic layer. The total yield was 190 mg (76 wt %).

### ADDITIONAL EMBODIMENTS

1. A method comprising:
(a) reacting a lignin comprising a 1,3 diol with carbon dioxide, a first catalyst, and an activating agent, in a first solvent to provide a lignin-monomer comprising a six-atom cyclic carbonate;
(b) reacting the lignin-monomer comprising a six-atom cyclic carbonate with a second catalyst, initiator, or combination thereof, in a second solvent to provide a polycarbonate.
2. A method comprising: reacting a lignin comprising a 1,3 diol with carbon dioxide, a first catalyst, and an activating agent, in a first solvent to provide a lignin-monomer comprising a six-atom cyclic carbonate.
3. A method comprising reacting a lignin monomer comprising a six-atom cyclic carbonate with a second catalyst, initiator, or combination thereof, in a second solvent to provide a polycarbonate.
4. The method of any of embodiments 1-3, wherein the lignin is reacted with carbon dioxide and the first catalyst to form a first intermediate, and the first intermediate is combined with an activating agent.
5. The method of any of embodiments 1-4, wherein the first catalyst comprises a cerium catalyst, a tin catalyst, an organometallic catalyst, a tertiary amine catalyst, or a combination thereof.
6. The method of any of embodiments 1-5, wherein the first catalyst comprises CeO₂, CeO₂—ZrO, Bu₂Sn(OCH₃)₂, AgOAc, ZnI₂/NEt₃, 1,8-diazabicyclo(5.4.0) undec-7-ene or a combination thereof.
7. The method of any of embodiments 1-6, wherein the first catalyst comprises 1,8-diazabicyclo(5.4.0) undec-7-ene.
8. The method of any one of embodiments 1-7, wherein the lignin has a weight average molecular weight (M_{w}) from 10,000-25,000 g/mol, 25,000-50,000 g/mol, 10,000-50,000 g/mol, 1,000-10,000 g/mol, from 1,000-5,000 g/mol, from 1,000-2,000 g/mol, from 1,000-3,000 g/mol, from 1,000-4,000 g/mol, from 2,000-5,000 g/mol, from 2,000-4,000 g/mol, from 2,000-3,000 g/mol, from 3,000-5,000 g/mol, or from 4,000-5,000 g/mol.
9. The method of any of embodiments 1-8, wherein the activating reagent comprises a dihalide, alkyl halide, sulfonyl halide, acid chloride, or a combination thereof.
10. The method of any of embodiments 1-9, wherein the activating reagent comprises a sulfonyl halide and a tertiary amine base.
11. The method of any of claim 1-10, wherein the activating reagent comprises methane sulfonyl chloride, trifluoromethanesulfonyl chloride, or toluenesulfonyl chloride.
12. The method of any of claim 1-11, wherein the first solvent has a dielectric constant (ε) (at 20° C.) of the solvent is at least 25, at least 30, or at least 35.
13. The method of any of embodiments 1-12, wherein the first solvent comprises dimethylacetamide, dimethylformamide, acetonitrile, dimethylsulfoxide, or a combination thereof.
14. The method of any of embodiments 1-13 wherein the lignin monomer is reacted with a second catalyst comprising an organometallic compound, Lewis acid, Bronsted acid, amine, or combination thereof, in a second solvent.
15. The method of any of embodiments 1-14, wherein the lignin monomer is reacted with an aminidine, guanidine, tertiary amine, or combination thereof.
16. The method of any of embodiments 1-15, wherein the lignin monomer is reacted with 1,5,7-triazabicyclo[4.4.0]dec-5-ene.
17. The method of any of embodiments 1-16, wherein the lignin monomer is reacted with an alcohol, a carboxylic acid, or a combination thereof.
18. The method of any of embodiments 1-17, wherein the lignin monomer is reacted with a Bronsted acid.
19. The method of any of embodiments 1-18, wherein the lignin monomer is reacted with a Lewis acid.
20. The method of any of embodiments 1-19, wherein the second solvent has a dielectric constant (ε) (at 20° C.) that is no greater than 25, no greater than 20, no greater than 15, no greater than 10, or no greater than 5.
21. The method of any of embodiments 1-20, wherein the second solvent comprises dichloromethane, toluene, ethyl acetate, n-butyl acetate, glyme, methylethylketone, acetone, or a combination thereof.
22. The method of any of embodiments 1-21, wherein the polycarbonate has a weight average molecular weight (as measured by GPC) that is from 50-500 kDa, from 100-500 kDa, from 100-300 kDa, from 200-300 kDa, from 150-200 kDa, from 150-175 kDa, from 175-200 kDa, from 150-250 kDa, from 200-250 kDa, from 200-225 kDa, from 200-250 kDa, or from 225-250 kDa.
23. The method of any of embodiments 1-22, wherein the polycarbonate has a polydispersity from 1-2, from 1-1.5, 1-1.25, from 1.25-1.5, from 1.5-1.75, or from 1.75-2.
24. A lignin monomer prepared according to the process of any of embodiments 2-13.
25. A lignin monomer, having the formula:

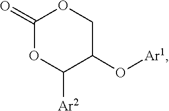

wherein Ar¹ and Ar² are independently an aromatic ring in a lignin structural moiety.
26. The lignin monomer according to claim 24 or 25, wherein the monomer has a weight average molecular weight (M_{w}) from 10,000-25,000 g/mol, 25,000-50,000 g/mol, 10,000-50,000 g/mol, 1,000-10,000 g/mol, from 1,000-5,000 g/mol, from 1,000-2,000 g/mol, from 1,000-3,000 g/mol, from 1,000-4,000 g/mol, from 2,000-5,000 g/mol, from 2,000-4,000 g/mol, from 2,000-3,000 g/mol, from 3,000-5,000 g/mol, or from 4,000-5,000 g/mol.
27. A polycarbonate prepared according to a process of any of embodiments 1 or 2-23.
28. A lignin-based polycarbonate, comprising units having the formula:

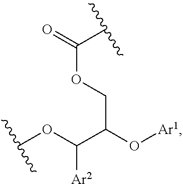

wherein Ar¹ and Ar² are independently an aromatic ring in a lignin structural moiety.
29. The polycarbonate of claim 28, wherein the polycarbonate has a weight average molecular weight (as measured by GPC) that is from 50-500 kDa, from 100-500 kDa, from 100-300 kDa, from 200-300 kDa, from 150-200 kDa, from 150-175 kDa, from 175-200 kDa, from 150-250 kDa, from 200-250 kDa, from 200-225 kDa, from 200-250 kDa, or from 225-250 kDa.
30. The polycarbonate of claim 28 or 29, wherein the polycarbonate has a polydispersity from 1-2, from 1-1.5, 1-1.25, from 1.25-1.5, from 1.5-1.75, or from 1.75-2.
31. A method of recycling a lignin-based polycarbonate, comprising combining a lignin-based polycarbonate with a recycling catalyst in a recycling solvent.
32. The method according to claim 31, wherein the recycling catalyst comprises cerium catalyst like CeO₂ or CeO₂—ZrO (mixite) oxide catalysts, a tin catalyst such as Bu₂Sn(OCH₃)₂, an organometallic catalyst such as copper-based catalysts, magnesium-based catalysts, aluminum-based catalysts, silver-based catalysts, for example, AgOAc, zinc-based catalysts, such as ZnI₂/NEt₃, cobalt-based catalysts, chromium-based catalysts, iron-based catalysts, nickel-based catalysts, titanium-based catalysts, hafnium-based catalysts, and zirconium-based catalysts, a tertiary amine catalyst such as 1,8-diazabicyclo(5.4.0) undec-7-ene (DBU), or any combination thereof.
33. The method according to claim 32, wherein the recycling solvent dimethylacetamide, dimethylformamide, acetonitrile, dimethylsulfoxide, or a combination thereof.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A lignin-based polycarbonate, comprising units having the formula: 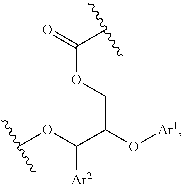
wherein Ar¹ and Ar² are independently an aromatic ring in a lignin structural moiety.

2. The polycarbonate of claim 1, wherein the polycarbonate has a weight average molecular weight (as measured by GPC) that is from 50-500 kDa.

3. The polycarbonate of claim 1, wherein the polycarbonate has a polydispersity from 1-2.

4. A method, comprising:
reacting a lignin comprising a 1,3 diol with carbon dioxide, a first catalyst, and an activating agent, in a first solvent to provide a lignin-monomer comprising a six-atom cyclic carbonate; and
reacting the lignin monomer comprising a six-atom cyclic carbonate with a second catalyst, initiator, or combination thereof, in a second solvent to provide a polycarbonate.
